(19) United States
(12) Patent Application Publication
(10) Pub. No.: US 2003/0154105 A1
(43) Pub. Date: Aug. 14, 2003

(54) Systems and methods for obtaining data correlated patient samples

(76) Inventor: Martin L. Ferguson, Medford, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/316,164

(22) Filed: Dec. 10, 2002

Related U.S. Application Data
(60) Provisional application No. 60/340,117, filed on Dec. 10, 2001.

(51) Int. Cl. G06F 17/60; G06F 7/00; G06F 17/30; G06F 19/00; G01N 33/48; G01N 33/50

(52) U.S. Cl. 705/2; 707/3; 702/19

(57) ABSTRACT

Systems and methods for allowing researchers to obtain well characterized, high quality patient samples and/or associated clinical information are provided.

### RELATED APPLICATIONS

This application claims the benefit of priority to U.S. patent application Ser. No. 60/340,117, filed Dec. 10, 2001.

### BACKGROUND

Various technologies developed within recent years have been useful for studying genetic mutations, aberrant gene expression patterns and faulty protein interactions that cause or contribute to disease. A better understanding of the molecular basis of many diseases has resulted in the identification of gene and protein targets (markers), that have been useful for developing novel therapeutics and diagnostics.

Although biological materials obtained from patients have been used in these studies, such samples have not been available in the quality and quantity to support genomics-scale experiments. Furthermore, the correlation of patient samples with broad, structured clinical information about the patient would greatly increase the value of these materials for research. (Skjei E, “Arraying the data,” CAP Today, March 2001, www.CAP.org/HTML/publications/archive, downloaded Aug. 16, 2001). In addition to the clinical information, the availability of pathological information that characterizes the material would allow a researcher to better define particular material that would be useful in a certain line of research.

However, a number of hurdles have impeded the availability of high quality biological materials associated with patient clinical information. For example, effective correlation of the materials and the patient record has been frustrated by concerns over patient privacy. In addition, the availability of various samples has been hampered by the fact that written “informed consents” must be obtained from patients. In addition, pathological information that would be of interest to a researcher, may not be obtained by a pathologist, who is primarily concerned about supporting the effective treatment of a patient.

Systems and methods for providing researchers with statistically significant numbers of high quality patient samples that are associated with clinical information are needed.

### SUMMARY

In one aspect, the invention features computer implemented methods for providing biological materials (such as tissues, cell or cell containing specimens) or derivative products (such as isolate cells, cell sections, tissue section, histocores or cellular components (e.g. DNA, RNA, protein, lipids, etc)) to a user. In one embodiment, the method comprises the steps of: receiving a user query, which identifies at least one desired characteristic of the biological material; identifying a biological material that has the at least one characteristic; receiving specification of the format for the biological material; and providing to the user, the at least one biological material or derivative product, in the specified format.

In another aspect, the invention features computer implemented methods for providing clinical data, which is associated with a particular patient sample to a user. In one embodiment, the method comprises the steps of: (a) receiving from a user a query, which identifies at least one desired characteristic of the biological material; (b) identifying biological material that has the at least one characteristic; and (c) providing to the user the clinical data associated with the biological material identified in step (b).

Use of the instant claimed methods better enable researchers to identify and obtain appropriate patient samples for particular studies. In addition access to the patient sample correlated clinical information and sample-level-information can be useful for the performance of statistical studies or for making or confirming a diagnosis.

Other features, objects and advantages of the invention will be apparent from the following figures, detailed description and claims.

### BRIEF DESCRIPTION OF THE FIGURES

FIG. 1 is a diagram of a system for providing access to biological material.

FIG. 2 is a diagram of a system for providing access to clinical data.

FIG. 3 is a screenshot of a user interface for generating a query for samples meeting query criteria.

FIG. 4 is a flow-chart of a process for making biological material available to a user.

FIG. 5 is a flow-chart of a system for obtaining biological material based on a request.

FIGS. 6-13 are flow-charts of a sample system for acquiring, storing, and delivering biological material.

### DETAILED DESCRIPTION

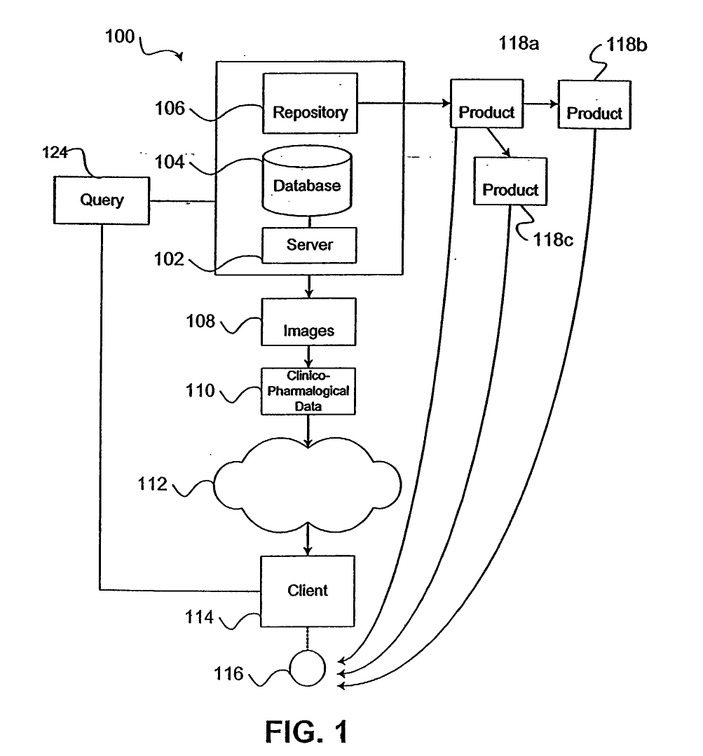

FIG. 1 illustrates an example of a system 100 that allows a user 116 operating a client 114 (e.g., a computer) to access information stored in a biological material database 104 over a network 112. The database 104 can include images 108 of physically stored biological material in a repository 106 and corresponding clinical data 110. The system 100 can permit a user 116 to specify characteristics and identify biological material of interest within the repository 106, view corresponding images 108 of the material, access associated clinical data 110, and/or order biological materials 118 (a) or derivative products 118 (b) or arrays of biological materials or derivative products 118 (c).

The clinical data 110 may be stored in conjunction with the material in the repository 106 or may be added in subsequently, for example to report a patient outcome. Biological materials can include tissue, cells or cell containing specimens, which have been prepared by any of a variety of means (e.g. fresh, frozen or paraffin embedded). These biological materials can be further processed into derivative products 118b such as tissue sections or histocores, cell sections, isolated cells or cellular components. Cells and cellular components (e.g. DNA, RNA, proteins, lipids, carbohydrates, etc.) can be extracted from tissues or cells using any of a variety of well-known techniques, including laser capture microscopy. In addition, the biological material or derivative products can be provided in conjunction with other materials as an array 118(c). For example, histocores of tumor tissue from different patients who have stage IV colon cancer can be provided as an array.

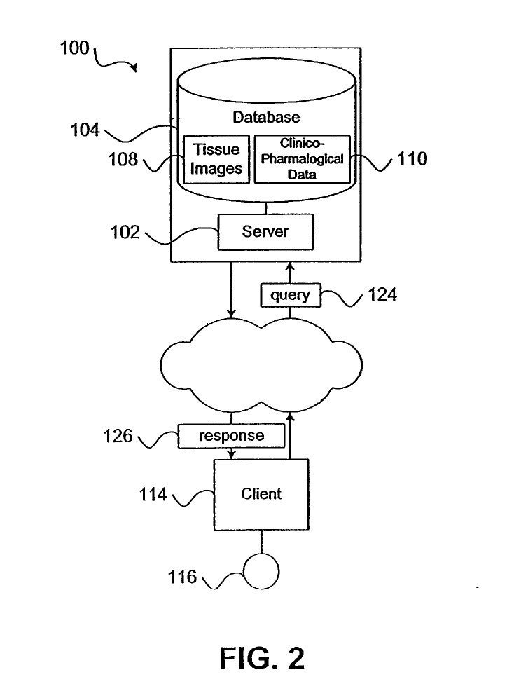

Although FIG. 1 shows a single user 116/client 114, the system 100 can readily provide countless numbers of users at remote locations access to the repository 106 and/or database 104. The provision of images 108, clinical data 110 and biological material products 118(a)-(c) to the user 116 takes place in response to a query 124 input into the system 100 by the user 116. This process is further illustrated in FIG. 2.

As shown in FIG. 2, a user 116 can submit a query 124 or request to a server 102 over the network 112. The query 124 may include Boolean terms and connectors or other querying techniques. After accessing the database 104 to identify biological materials 122, images of biological materials 108, and/or clinical data 110 matching the query 124, the server 102 can transmit a query response 126 back to the user's 116 client computer 114. For example, the response 126 may feature a dynamically constructed web page that includes information about the number of biological materials available that correspond to the query 124 parameters, including projections about the statistical adequacy of available numbers of biological materials for powering a particular research protocol. The dynamically constructed web page may offer prompts or menus triggered by the number or type of samples available that correspond to the query 124. The response 126 may call for more information to be input by the user 116, which may then be formed as a follow-on query 124. The system can respond to this follow-on query by refining the selection of tissue samples and by providing this refined selection to the user 116. In addition to information about available biological materials corresponding to the user's 116 query 124, the system may respond to an appropriate query by providing clinical datasets that are associated with the biological materials. As one form of output, the system may provide ordered arrangements of clinical datasets in addition to or independent of the biological materials or derivative products. Alternatively, the response 126 may present appropriate images 108 or clinical data 110, for example, that can be accessed for performing statistical analyses on the tissues, for facilitating further refinement of the query 124 or for later obtaining outcome information on the patient from whom the sample had been obtained. As shown in this figure, interplay between user query 124 and system requirements 126 permits the user 116 to assemble customized arrays of biological products.

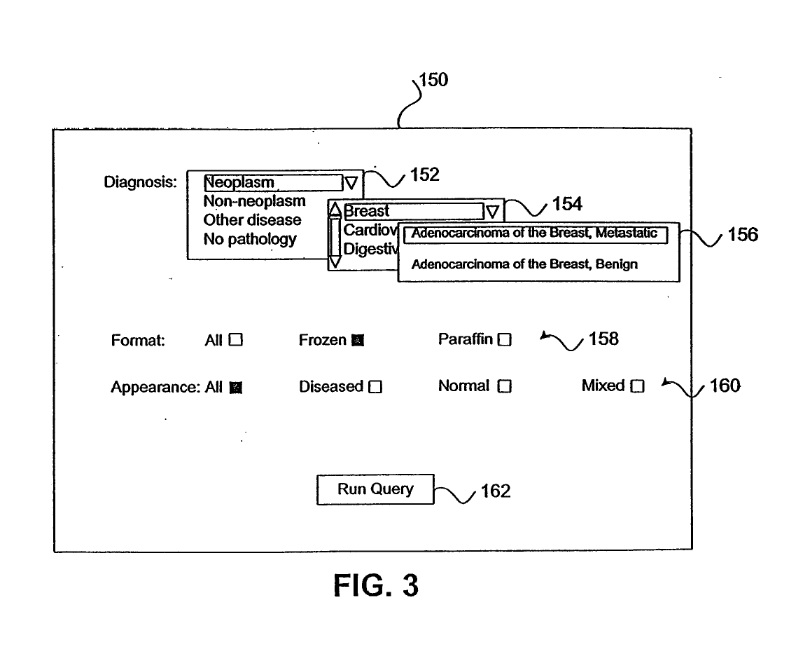

FIG. 3 illustrates a user interface 150 that enables a user to prepare a query by navigating through a series of text description menus 152-156. For example, the menus shown enable the user to select different characteristics of a patient sample (e.g., whether the diagnosis is neoplastic 152 and the type of tissue sample 154). The user interface 150 may also include other “widgets”. For example, the interface 150 may enable a researcher to specify a format 158 and/or a tissue appearance 160. After selecting a diagnosis 156 and specifying other attributes 158, 160, a user may submit a corresponding query 162.

The user interface shown in FIG. 3 may be expressed in instructions for transmission over the Internet to a user's client (e.g., web-browser). For example, the instructions may feature markup language instructions such as HTML (HyperText Markup Language), XML (eXtensible Markup Language), or another SGML (Standard Generalized Markup Language) language. Such instructions may be transmitted via a network protocol such as HTTP (HyperText Transfer Protocol) and/or HTTPS (HyperText Transfer Protocol Secure).

The user interface shown in FIG. 3 is merely an example. Other user interfaces may incorporate images of biological material. For example, a user interface may present an image and enable a user to request other samples sharing characteristics with the displayed image. Additionally, the user interface may permit a user to specify a selection of the image, such as a selected cell, for inclusion in a product 118a. One example of techniques for providing tissue images for web-based image analysis is provided in Bova et al., “Web-based tissue microarray image data analysis: initial validation testing through prostate cancer Gleason grading,” Hum. Pathol. 32:417-427, 2001, the contents of which is incorporated herein by reference.

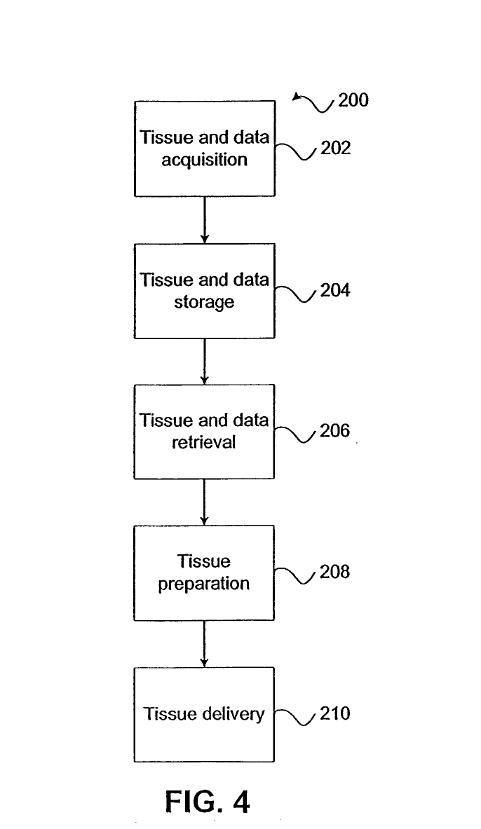

FIG. 4 illustrates a flow-chart of an exemplary process 200 for making biological material available to users. The process 200 stores 204 biological material and corresponding data received 202 from donor institutions. The process 200 enables users to select tissue for retrieval 208 and can deliver 210 the tissue in a user specified form 208. Each of these tasks is described in greater detail below.

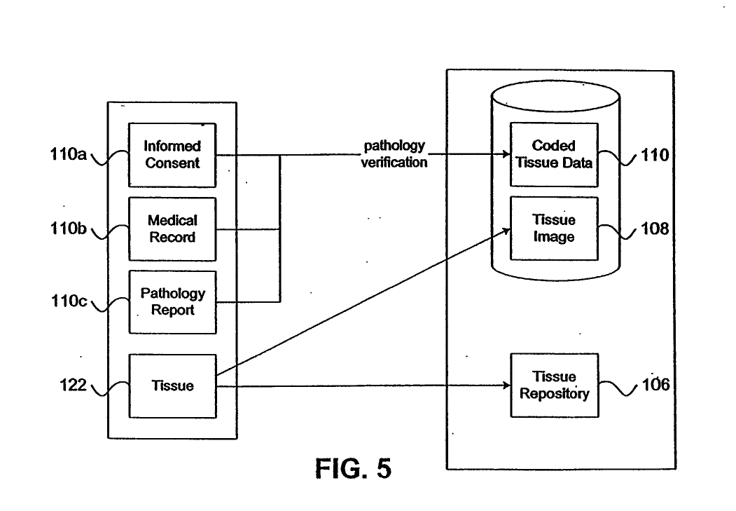

FIG. 5 shows generally a process by which qualified donor institutions 120 can provide excess biological material 122 for processing and storage within the tissue repository 106. Images 108 of the materials 122 may be obtained and coded in a manner which correlates the image 108 with the material 122 in the repository 106. In addition, to the image(s) 108, the database 104 can include related clinical data 110 that has been abstracted, for example, from information provided from donor institutions or developed from subsequent analysis of the materials. Such data 110 may include familial information, clinical history, medications, disease and treatment history, demographic information, laboratory reports, pathology and histology reports, outcome reports, molecular profile data (RNA expression, protein expression, metabolite levels) and so forth. The clinical data 110 is also coded in a manner, which correlates the data 110 with the materials 122 in the repository 106. Information supplied from donor institutions 120 can include, for example: (1) confirmation 110a of the existence of an informed consent from the patient from whom the tissue has been obtained or the actual informed consent document; (2) relevant clinical information 110b about the patient; and/or (3) a pathology report 110c, which characterizes the tissue. In certain embodiments of the system, the presence of an informed consent 110a may be a necessary datum without which no other coded tissue data may be stored in the database 104.

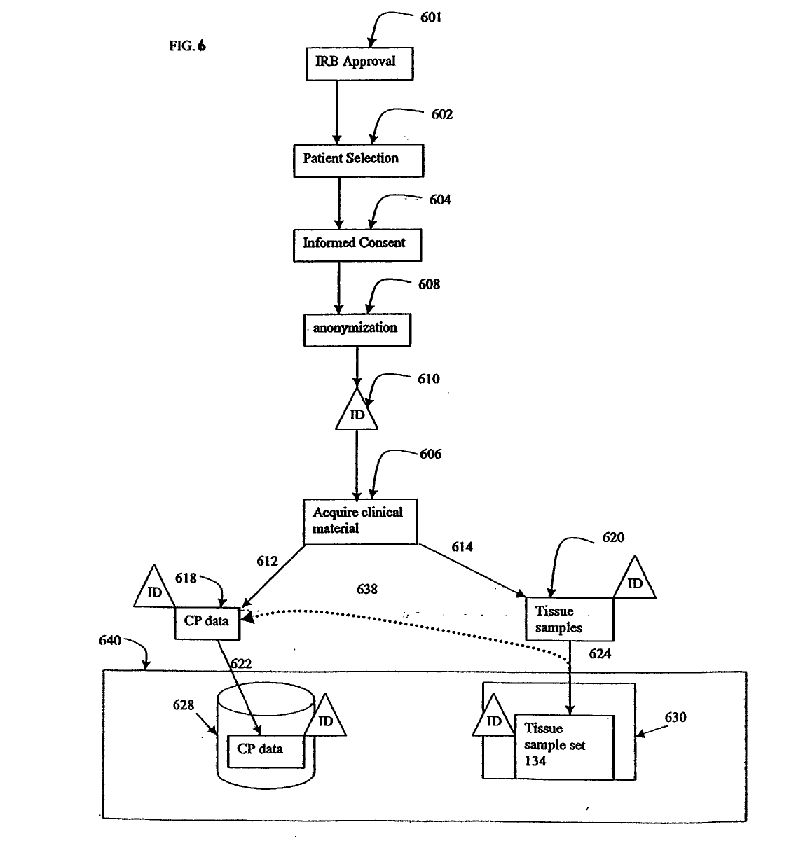

FIG. 6-13 illustrate examples of systems and methods that allow for the assembly of a searchable library of human biological materials and associated clinical information. As shown, the materials and information are collected in conformity to rigorous bioethical constraints, including IRB approval procedures and mechanisms for obtaining informed consent, and further including anonymization procedures that disassociate the materials and information from any personal information that could be used to identify the donor of these materials and information. Certain of these processes are illustrated in FIG. 6.

FIG. 6 is a flow chart illustrating an exemplary process 600 for assembling a collection of tissue samples and related clinical data in such a way that these samples and data are retrievable from a storage area. Initially, a protocol for collection of tissues and data is submitted for IRB approval, and IRB approval 601 is obtained. Next, patient selection 602 is performed. Criteria for patient selection 602 may be specified within the system, so that patients likely to be harboring certain types of pathological tissues or patients fitting into certain medical or demographic categories are preferentially sought out for inclusion. Patients may be selected in accordance with predetermined criteria, such as present disease state, past disease state, anticipated pathological condition, demographic characteristics, or any other selection criteria that are presently or may be subsequently determined to have utility for selecting tissue donors. A plurality of patients may be selected from at least two health care institutions; selected patients will be limited to those who will imminently undergo tissue removal at the health care institution. The selected patients may also be termed tissue donors or tissue sources, terms that refer to a person with intact or preserved cardiorespiratory function from whom surplus clinical material is available. As used herein “surplus clinical material” may refer to any human biological material that is initially removed by a diagnostic or therapeutic procedure but is not subsequently needed for diagnostic or therapeutic purposes. Tissue removal is understood to include any type of removal, including surgical excision, needle biopsy, fluid extraction, or any other sort of removal procedure that will be familiar to practitioners in the art.

After patient selection 602, a legally appropriate informed consent is obtained 604 from the selected patient. A process of anonymization 608 is then performed, whereby a unique identifier is assigned to the tissue donor so that the anonymity of the tissue donor is safeguarded. This unique identifier may be a coded symbol, alphanumeric or other, that provides reference to the individual tissue donor and to the transaction from which surplus clinical material is being obtained, while still safeguarding the anonymity of the tissue donor. A number of processes for anonymization are available and will be familiar to those of ordinary skill in the relevant arts. This anonymization process results in the creation of a unique identifier 610 that can subsequently be used to identify tissues and data derived from a tissue donor in such a way that no personal information about the tissue donor can be associated with the tissues or data derived therefrom. Components of personal information may include identifying characteristics such as patient name, Social Security number, address, phone number, or any other individually associated information that can be used to identify the tissue donor. The unique identifier 610 may then be applied to clinical materials 606 derived from the tissue donor. Clinical materials 606 include clinical information and human biological material. These two different types of clinical materials 106 are processed differently, represented schematically in FIG. 6 by the clinical information processing arm 612 and by the biological material processing arm 614.

As a result of processing 612 clinical information, clinical data 618 is produced, shown here to be inextricably linked to the unique identifier. Clinical information is contained in the medical record of the tissue donor and can be extracted therefrom, codified, arranged or processed to produce clinical data 618. Data may be extracted from the tissue donor's medical record manually by data gathering personnel who complete, for example, fields set forth on a clinical data entry form by referring to the tissue donor's hard copy medical record. Data may also be extracted from a medical record electronically or by searching techniques or by any other technique of data extraction available in the art. These clinical data 618 may then be subjected to further data processing, as shown at step 622. After processing, the processed clinical data may be entered into a database 628 still in association with its unique identifier. Other clinical data may be generated during tissue sample processing when these samples acquired from the tissue donor are examined by an independent pathologist. Data produced through such pathological examination may then be added to those clinical data 618 acquired from the medical record, as shown by arrow 638.

Processing for human biological materials is represented diagrammatically by arrow 614. The human biological material removed from the tissue donor undergoes further processing 614 to yield tissue samples 620, all associated with the unique identifier. After additional tissue processing 624, described in more detail below, a set of tissue samples 632 associated with the unique identifier 610 may be retained and preserved in a biorepository 630.

The unique identifier 610, also associated with clinical data in the database 628, permits these data to be associated relationally with the tissue sample set 632 stored in the biorepository 630 that is associated with the same unique identifier 610. In this way, reference to the clinical data may provide access to the correlated tissue samples and vice versa, as will be described in more detail below. The database 628 and the biorepository 630 may together be considered a storage area 640 for data and tissue samples collected according to the systems and methods of the present invention.

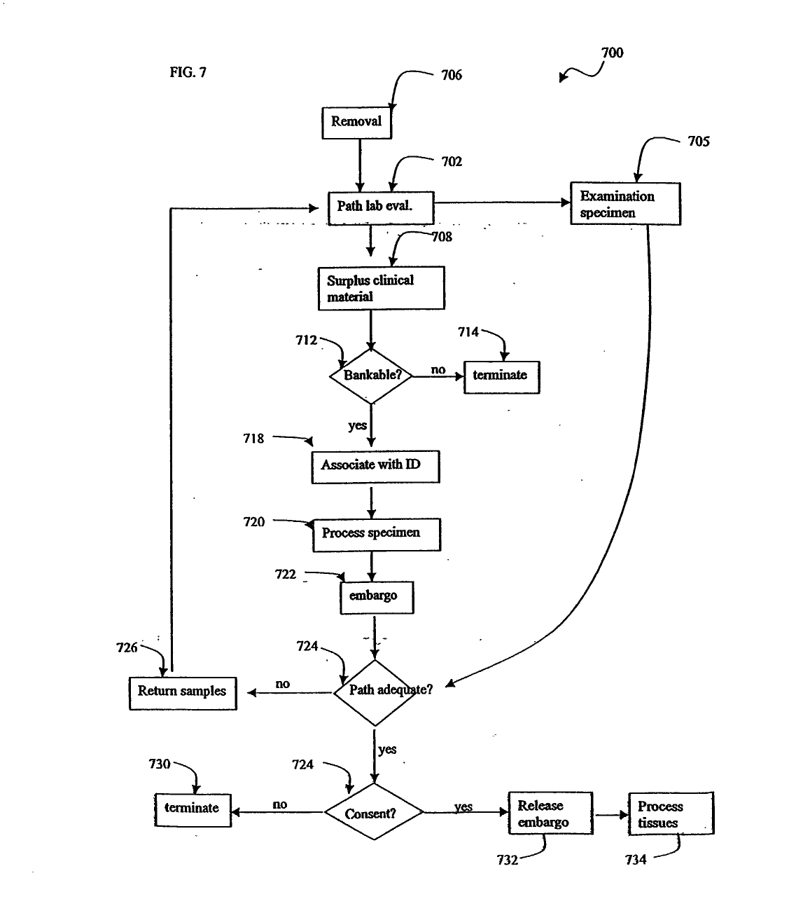

FIG. 7 shows in more detail a process by which samples may be acquired from a tissue donor. Initially, human biological materials are removed 706 from the tissue donor. This takes place after certain procedures described previously in FIG. 6 have been performed, including IRB approval of the acquisition protocol, patient selection, and obtaining legally appropriate informed consent. After removal 706 of human biological materials, these materials are evaluated 702 in the health care institution's pathology lab, in accordance with standard procedures. During pathology lab evaluation 702, the human biological material is split into an examination specimen 704 and surplus clinical material 708. Processing the examination specimen 704 results in a pathology report that is a source for pathology data 710 that may be included in the donor-related clinical data, as described above. Furthermore, the examination specimen 704 is evaluated by the health care institution pathologist, who decides whether the examination specimen 704 is adequate for the patient-related diagnosis 724 that the pathologist needs to provide. The results of this decision 724 will determine whether tissue samples derived from the surplus clinical material 708 are released from embargo 722 or are returned to the health care institute for further analysis, as will be discussed below in more detail.

The arrow 708 in FIG. 7 shows a path for processing the surplus clinical materials that are obtained. Decision point 712 requires the determination of whether the surplus clinical materials represent bankable specimens. Criteria for bankable specimens may include the physical condition of the surplus clinical material, its likelihood of containing useful tissue types or any other criteria that may be identified and applied to the surplus clinical material. As shown at 714, if the surplus clinical materials do not include bankable specimens, no further procedures are undertaken. If, however, the surplus clinical materials do include bankable specimens, they are thereupon associated 718 with a unique identifier. The specimen is then processed 720 to yield tissue samples. These tissue samples are embargoed 722 to await the results of pathology adequacy verification and clinical diagnosis at 724. As described above, pathology adequacy verification is the process by which the health care institution pathologist determines whether the specimen available for pathological analysis is adequate for diagnostic or therapeutic purposes. If the specimen available for pathological analysis is not considered adequate, the tissue samples and residual surplus clinical material are returned to the pathology lab 726 to undergo further pathological evaluation 702. If pathology adequacy verification is satisfactory, the informed consent status of the tissue donor is then verified 728. As shown at 730, if a legally appropriate informed consent status cannot be verified, no further procedures will be undertaken. If the informed consent status can be verified and the clinical diagnostic process is complete, the tissue samples are released from embargo as shown at 732. Further tissue processing 734 then takes place, as will be described in more detail below.

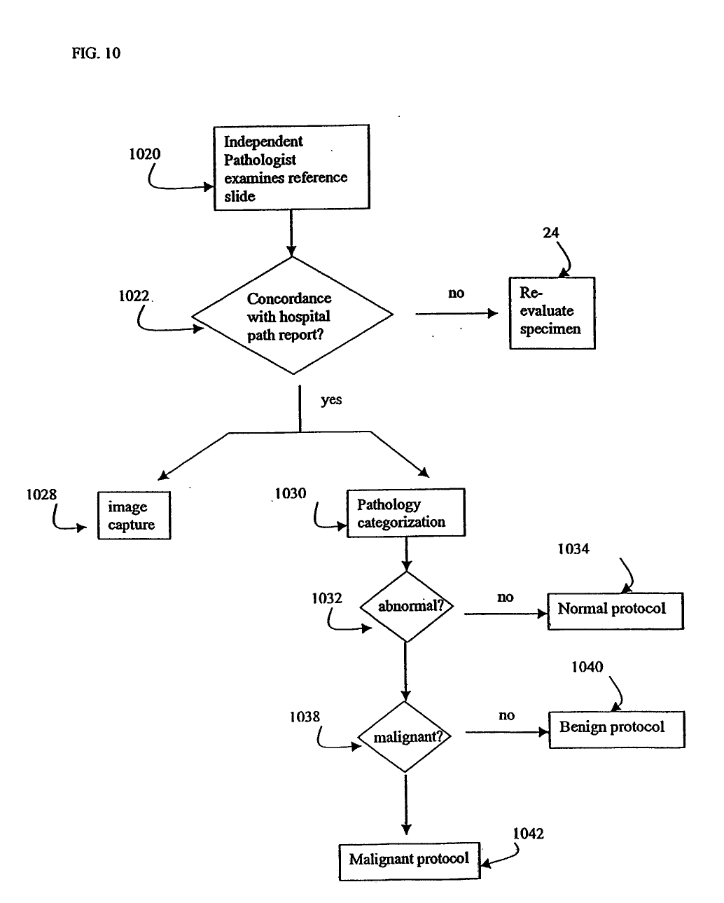

In addition to the pathology data produced by the health care institution that may subsequently be stored in a database in relation to processed tissue samples, a separate set of sample-related pathology data is produced for each sample when it is examined by an independent pathologist not related to the health care institution. A flow diagram showing exemplary steps of this process is provided in FIG. 10. Initially, an independent pathologist examines 1020 a reference slide that is representative of a tissue sample or set of tissue samples. Such a reference slide may be produced according to those methods set forth in Example 4 below, or may be produced by following other techniques familiar to skilled artisans. After the reference slide is examined, the pathologist will compare her diagnostic conclusions with those presented on the hospital's pathology report that was produced when the hospital pathologist examined the specimen associated with the human biological material removed from the tissue donor. As an example, the independent pathologist may determine whether there is concordance 1022 between diagnostic features contained in the hospital pathology report and those she identified through her examination of the reference slide. The independent pathologist may consider such diagnostic features as gross appearance and top line pathology diagnosis, although other diagnostic features may also be considered. The concordance step 1022 may result in a conclusion of no concordance, in which case the suitability of the tissue samples for further processing and storage may be reevaluated 1024. If concordance is confirmed, further characterization 1030 of the microscopic features of the reference slide may be carried out. In addition, a set of digital images may be captured 1028 from representative portions of the reference slide, to be used for subsequent correlations between tissue specimens and related clinical data.

When the independent pathologist characterizes the features of the reference slide, she may evaluate the composition of the tissues to determine whether they are abnormal 1032. If they are not abnormal, their features will be characterized according to a normal protocol 1034. If abnormal, the tissues may be further evaluated to determine whether they are malignant 1038. If they are not diagnosed to be malignant, their features may be characterized according to a benign protocol 1040. If they are malignant, their features may be characterized according to a malignant protocol 1042. Each of these aforesaid protocols relates to a set of characteristics that may be used to describe a particular tissue type, whether normal, benign, or malignant. A particular normal protocol 1034 may exist, for example, for each type of normal tissue or organ whereby descriptive data is collected. In each such protocol, a list of descriptors may be drawn up by an expert in that particular diagnostic area; the independent pathologist may then determine the presence or absence of these descriptors in the particular tissue specimen that she is examining under that particular protocol. Similarly, for abnormal tissue afflicted with benign disease, a protocol 1040 may be drawn up that recites pathological features whose presence, absence or extensiveness the pathologist may evaluate when she is examining a particular specimen. Analogously, for abnormal tissue afflicted with malignant disease, a protocol 1042 may be drawn up in like manner. For example,-in malignant specimens, several factors may be evaluated: 1) the proportion of tissue containing viable tumor cells; 2) the proportion of tissue that is normal; and 3) other characteristics, such as presence of necrosis and cellularity of the stroma. Various categorization schemes are known in the art that list features and characteristics of normal and pathological tissues. Categorization schemes may be provided by standard nosological references such as SNOMED® and the College of American Pathologists Cancer Protocol Manual, “Reporting on Cancer Specimens,” although other nomenclature and classification resources may also be employed, as will be appreciated by those of ordinary skill in the art. Protocols for pathologic assessment of a particular tissue sample may be drawn from these categorization schemes or modifications thereof. In addition to protocol-driven evaluation of tissue samples, the independent pathologist may offer other comments or remarks as part of her examination process. In certain embodiments, these comments or remarks may provide the basis for additions to or revisions of existing protocols, or may give rise to the creation of new protocols. A set of protocols for categorizing pathological features of tissue samples is provided in the co-pending patent application Ser. No. 10/053,082, entitled “Encoding a Diagnosis”, which was filed on Nov. 2, 2001, the disclosure of which is incorporated herein by reference.

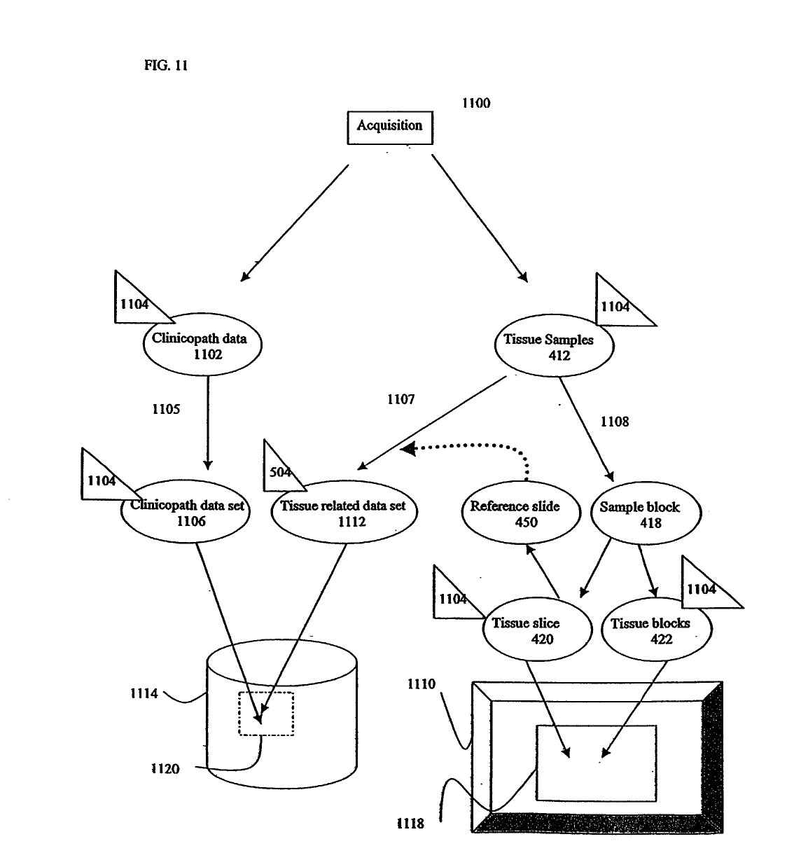

FIG. 11 shows schematically certain procedures for data and tissue storage according to the systems and methods of the present invention. Data and tissue acquisition 1100 may be performed in accordance with processes described above. The data and tissue acquisition processes 1100 as applied to a particular patient's human biological material and associated clinical information result in the acquisition of clinical data 1102 and tissue samples 412, each of which is tagged with an identifier 1104 that allows them to be related to each other. As previously described, the identifier 1104 is selected so as to preserve the anonymity of the patient from which the human biological material and associated clinical information is obtained. The identifier 1104 further functions as a retrievability tag so that tissues related to a set of clinical data may be retrieved by reference to data in that data sent, or so that clinical data related to a tissue set may be retrieved by reference to the tissue set.

The clinical data 1102 undergoes classification and categorization 1105 to produce a clinical data set 1106. As previously described, this clinical data set 1106 may contain ordered and categorized features derived from the individual patient's clinical information, and may be organized as data fields, datasets, data subsets, or any other organized and structured arrangement of data that can be envisioned by those of ordinary skill in the art. The clinical data set 1106 remains linked to the identifier 1104 so that its relation to the tissue samples 412 may be retained.

As illustrated in this figure, tissue samples 412 undergo physical preparation 1108 prior to being stored. This step of physical preparation 1108 first results in the creation of a sample block 418. The sample block 418 may then be further processed to yield a plurality of tissue blocks 422 and a tissue slice 420 related to the tissue blocks 422 from which a reference slide 450 may be made that is representative of the tissue samples 412. As previously described, this reference slide 450 may be examined by an independent pathologist. The results of this examination may be added to other tissue-related data, as shown by dotted arrow 1113, to form a tissue-related data set 1112.

In addition to the physical preparation step 508, data may be derived 1107 from the tissue samples that relates to them. For example, classification or categorization data pertaining to the tissue samples 412 may be determined. These data combine with data derived from examination of the reference slide to comprise a tissue-derived data set 1112, which contains ordered and categorized features related to the tissue samples 412. The tissue related data set 1112 may be organized as data fields, data sets, data subsets, or any other organized and structured arrangement of data that can be envisioned by those of ordinary skill in the art. The tissue related data set 1112 remains linked to the identifier 1104 so that its relation to the clinical data 1102 may be retained.

The clinical data set 1106 and the tissue related data set 1112 are both then stored in a database 1114, and are related to each other by reference to the identifier 1104. The aggregate of the clinical data set 1106 and the tissue related data set 1112 linked by the identifier 1104 as stored in the database 1114 may be termed the data profile 1120 for human biological material and associated clinical information derived from a particular patient. The tissue blocks 422 and the tissue slice 420, each linked to the identifier 1104, are stored in a biorepository 1110 using preservation techniques described previously, or other preservation techniques that are known or that may be devised by practitioners of ordinary skill in the relevant arts. The tissue blocks 422 and the tissue slice 420 for a particular tissue donor's human biological material, as stored in the biorepository 1110 and as accessible by the identifier 1104 may be termed a tissue module 1118.

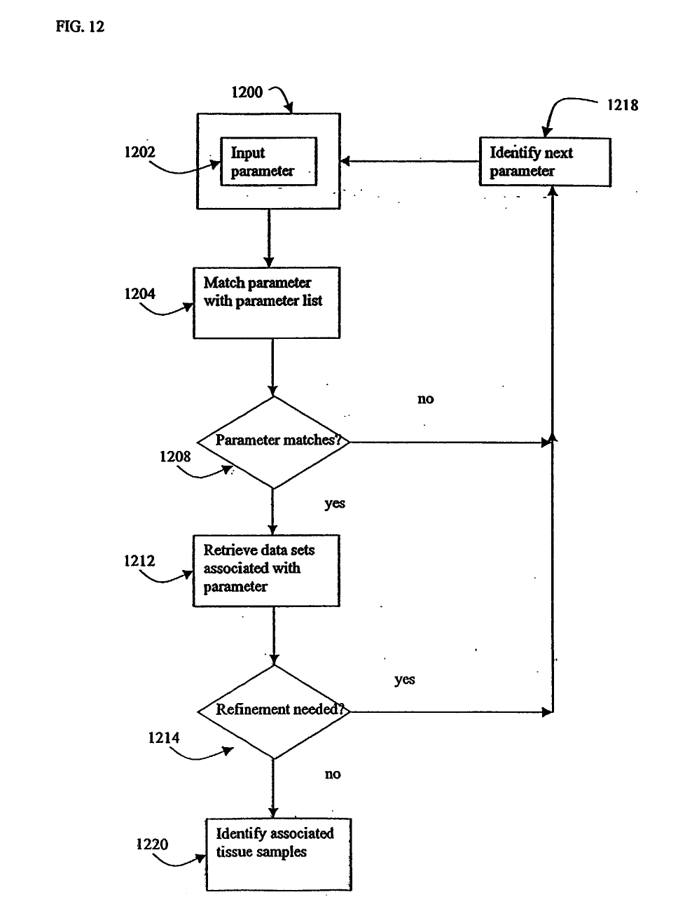

As shown in FIG. 12, a process 1200 is provided whereby tissues and data may be retrieved in response to parameters input according to which a selection of tissues or data may be chosen. As shown schematically in this figure, a user interface 1201 may be provided where a user (not shown) inputs a parameter 1202 according to which a set of, tissue samples or a set of clinical data may be retrieved. The user interface 1201 may be a computer terminal, a data entry device, or any other mechanism whereby textual, numerical or optical/digital data may be input. The parameter 1202 may take the form of a word, a set of words, a numerical code, an image or representation thereof, or any other imputable information that may be useful for retrieval of data or tissue samples. The parameter 1202 input by the user may be matched with parameters on a preselected list to evaluate its appropriateness and adequacy for tissue and data retrieval. Parameter match may then be evaluated by the system. If the parameter is determined to be acceptable, data sets are thereupon retrieved 1212 that are associated with the input parameter. The system may provide at that point an algorithm to evaluate, for example, the number of data sets retrieved, and to determine their quantity, quality and relevance. This algorithm may assist in determining 1214 whether further refinement of the retrieved datasets is needed. If the number of retrieved data sets is excessive, or if a number of apparently irrelevant data sets are retrieved in response to a particular input parameter, the system may determine that further refinement would be useful. If this further refinement is desirable, the system may request from the user a second parameter input, shown as step 1218. If a second input parameter is required, this parameter reenters the illustrated flowchart at step 1202. If, however, a second input parameter is not required, the system may proceed to identify those sets of tissue samples that are individually and uniquely associated with the selective data sets, as described in more detail below. Alternatively, at decision point 1208, if the parameter is determined not to be acceptable, the user is directed to identify a next parameter 1218 that may be prompted by the system in relation to the original parameter, so that the next parameter serves to refine the user's original parameter. For example, in response to an inappropriate parameter, the system may suggest to the user a set of parameters that are similar to the originally input parameter so that the user can select from the suggested set a parameter corresponding to the user's research needs and consistent with the scope of the parameters available to the system. Or, for example, in response to an inappropriate parameter, the system may provide a sequence of queries to which the user will respond that will allow the user to focus his selection of a parameter more usefully. These or other refinement processes may be available to guide selection of next parameters 1218 when the original parameter is unsatisfactory (decision point 1208) or when further data refinement is desirable (decision point 1214).

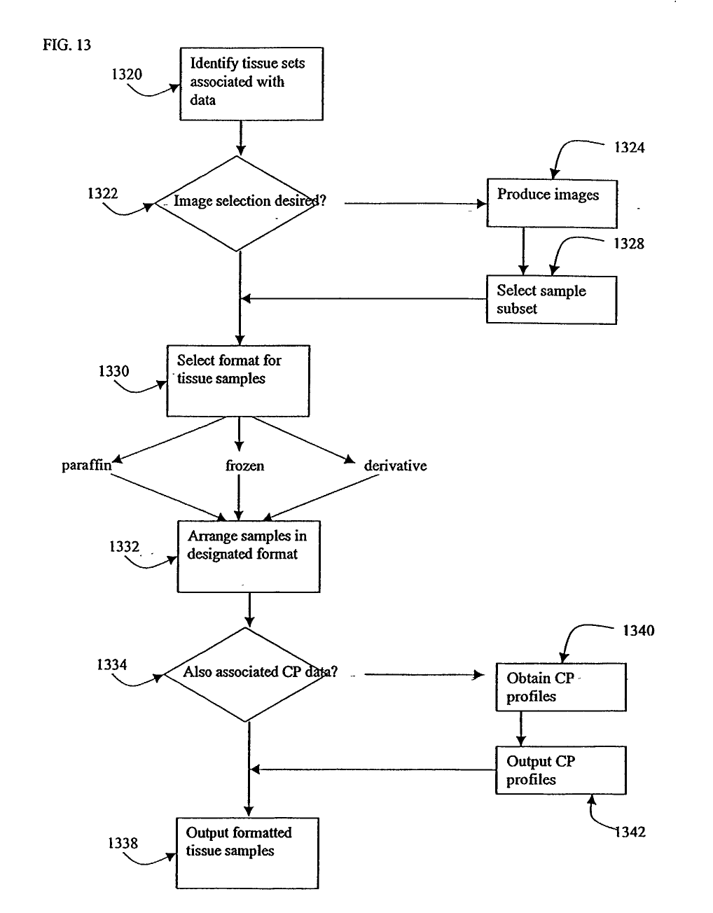

FIG. 13 shows further how the systems and methods of the present invention may interact to produce formatted sets of tissue samples. Step 1320 shows the identification of tissue samples associated with datasets, as described in the previous figure. At decision point 1322, the user determines whether image based selection is desired. If image based selection is desired, a set of images may be provided 1324 for the user's inspection that are associated with the tissue samples that have been previously identified at step 1320. The user may then select 1328 a subset of tissue samples with reference to the aforesaid images. Having a selected this subset, the user at step 1330 selects a format in which the tissue samples will ultimately be delivered to the user as a research product such as a microarray. With further reference to decision point 1322, if the user determines that image based selection is not desired, the user then proceeds to step 1330, the format selection step. As shown in this figure, several different formats for the tissue sample delivery may be selected. For example, the tissue samples may be delivered as a set of paraffin-preserved products. Alternatively, the tissue sample product may be delivered as a microarray of segments of paraffin preserved tissue samples. Or, for example, an assembly of derivative products from the selected tissue samples may be arranged. Derivative products may include RNA, DNA, proteins, small molecules, or any other biochemical component derived from or extracted from the selected tissue samples. Derivative products may include tissue samples subjected to additional processing techniques such as immunohistochemistry. After the product format has been selected in step 1330, the selected tissue samples are arranged in the designated format in step 1332. A variety of systems are available whereby the formatted tissue product may be fabricated. One example of a device useful for these processes is disclosed in Provisional Patent Application 60/306,741, “Instruments and Methods for Creating a Tissue Microarray,” to Chu and Chasse, filed Jul. 20, 2001, the contents of which are herein incorporated by reference. The user then may be asked whether an output of associated clinical data profiles is desired, as shown in step 1334. If this output is not desired, then the formatted tissue samples represents the final research product, as shown at step 1338. If associated clinical data profiles are desired, these are obtained and formatted as shown at step 1340. The final output then includes these associated clinical data profiles 1340, combined with the formatted tissue samples output 1338. In other embodiments, clinical data sets alone may form the final output product.

The present invention is further illustrated by the following examples which should not be construed as limiting in any way. The contents of all cited references (including literature references, issued patents, published patent applications as cited throughout this application) are hereby expressly incorporated by reference. The practice of the present invention will employ, unless otherwise indicated, conventional techniques that are within the skill of the art.

### EXAMPLES

### Example 1

Division of Surplus Clinical Material and Production of Tissue Samples

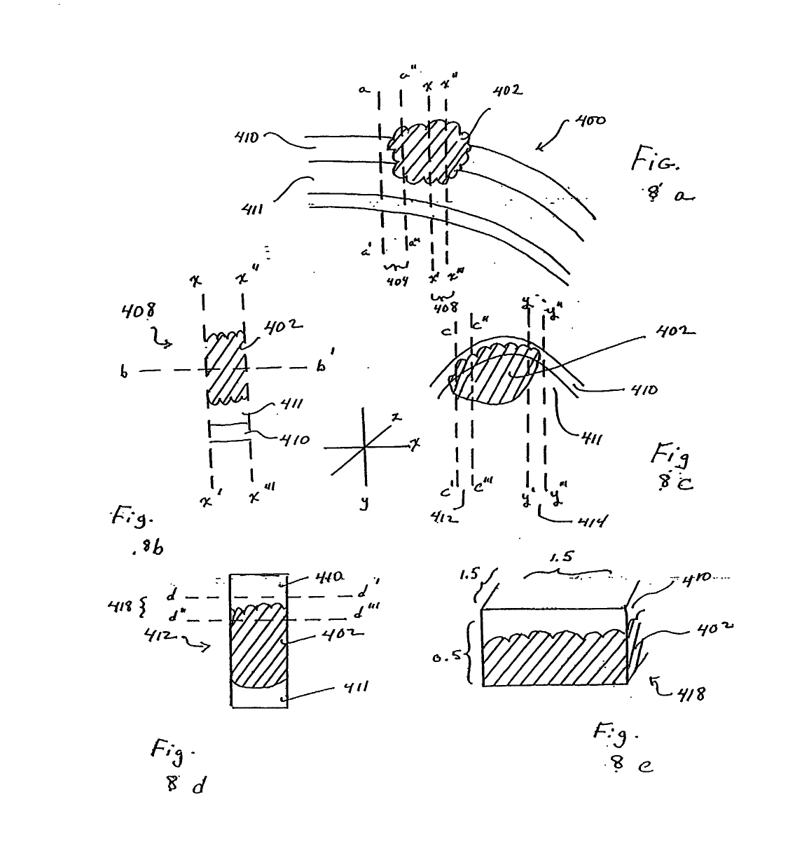

FIGS. 8a-e and 9a-c provide examples of processes for dividing surplus clinical material and producing tissue samples. The goal of these processes is to obtain both non-fixed and fixed portions of surplus clinical materials such that each non-fixed portion shares a cutting surface with a fixed portion. The shared cutting surface means that each non-fixed portion has a “mirror image” in a fixed portion. Any feature present on that surface of the fixed portion is necessarily present in the non-fixed portion. Therefore, the fixed portion can be examined to verify the presence of features in the non-fixed portion. This verification method does not require thawing or other potentially destructive manipulation of the non-fixed portion.

FIGS. 8a-e illustrate methods by which tissues may be further processed. FIG. 8a provides a diagram of a specimen 400 that has been removed from a tissue donor and sent to the health care institution pathology lab, as previously described. The specimen 400 may be a luminal structure as shown here containing a lesion 402 surrounded by normal tissue 410 and extending into a lumen 411. It is understood, however, that any type of surgical specimen 400 may be processed in accordance with these systems and methods. The specimen 400 is examined by a health care institution pathologist who identifies a section 404 (shown here to be the tissue segment between lines a-a′ and a″-a′″) to be evaluated for diagnosis. The remainder of the specimen 400 may be available for research purposes, including banking, and may be considered surplus clinical material. If the surplus clinical material is considered bankable, as described above, a research sample 408 (shown here to be the tissue segment between x-x′ and x″-x′″) may be excised from the specimen 400 for further preparation.

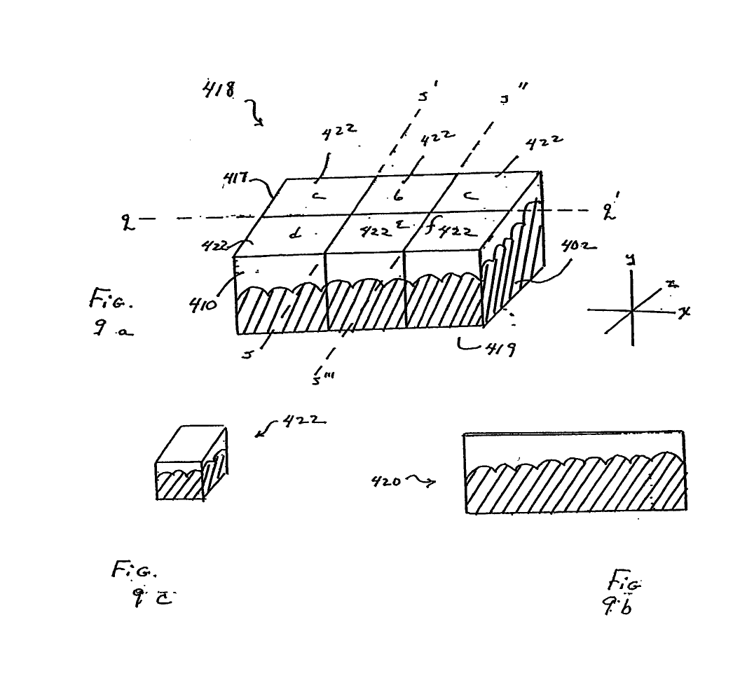

FIG. 8b shows in more detail one aspect of the research sample 408. This figure provides a longitudinal view of the research sample 408, showing schematically the lesion 402, the lumen 411 of the luminal structure, and an area of normal tissue 410. FIG. 8c shows a cross-section in the x-z plane of the research sample 408 taken at the line b-b′ in FIG. 8b. In FIG. 8c, the pathological lesion 402 is shown, extending into a lumen 411, and surrounded by a rim of normal tissue 410. With reference to this orientation, a tissue sample 412 or a tissue sample 414 may be excised. The tissue sample 412 is shown in more detail in FIG. 8d. FIG. 8d shows the orientation of the components of the tissue sample 412: the normal tissue 410 is depicted adjacent to the upper aspect of the lesion 402, with the lesion 402 protruding into a lumen 411. From this tissue sample 412 a sample block 418 is cut along the lines d-d′ and d″-d′″, and a shown in more detail in FIG. 8e. The dimensions of the sample block 418 are provided in FIG. 8e. The sample block 418 has a height of 0.5 cm, and a width and a length of 1.5 cm. The sample block 418 may then undergo further processing, as shown in FIGS. 9-c.

FIG. 9a shows the sample block 418 with an incision line indicated to extend along the lines q-q′ from a top surface 417 to a bottom surface 419 of the simple block. This incision provides a tissue slice 240 harvested in the x-y plane, from the top surface 417 to the bottom surface 419 shown in FIG. 9b. By making incisions into the sample block along the lines s-s′ and s″-s′″ a set of six tissue blocks 422 may be obtained. A representative tissue block 422 is shown schematically in the FIG. 9c. In FIGS. 9a-c, a portion of the lesion 402 and a portion of normal tissue 410 may be seen. In accordance with the aforesaid protocol, the tissue slice 420 may be preserved in paraffin. A reference slide (not shown) may be prepared from the surface of the tissue slice 420 that faces a set of tissue blocks 422. This reference slide thus may provide information about the structures on the tissue blocks 422 that face it. Each of the tissue blocks 422 is cryopreserved, as will be described in more detail below. Each tissue block 422 may be accompanied by identifying information, including its position with respect to the reference slide.

While FIGS. 8 a-f and 9 a-c illustrate aspects of the present invention as applied to a large volume specimen 400, the inventive methods of the present invention may be modified when applied to specimens of smaller volume. Appropriate configurations of tissue blocks 422 may readily be devised to account for these smaller volume specimens, as will be understood by those of ordinary skill in the art.

### EXAMPLE 2

Use of the System for Obtaining Materials for Breast Cancer Research

Ductal carcinoma-in-situ of the breast is a condition where cytologically malignant breast epithelial cells proliferate within the ducts but remain confined therein. It is understood that ductal carcinoma-in-situ is to be distinguished from atypical ductal hyperplasia and is further to be distinguished from invasive ductal carcinoma. Just as atypical ductal hyperplasia may progress to become ductal carcinoma-in-situ, so also may ductal carcinoma-in-situ progress to become invasive ductal carcinoma. This occurs in about one-third of untreated cases of ductal carcinoma-in-situ (DCIS).

Recognizing the likelihood of progression to invasive cancer, surgical oncologists traditionally have recommended total mastectomy as treatment for DCIS. This is paradoxical, however, now that wide excision with radiation is recognized as adequate treatment for early stage invasive breast cancer. The traditional recommendation of total mastectomy for the non-invasive condition of DCIS, although more aggressive than the standard treatment for early-stage invasive cancer, has been justified by the observation that following wide excision (with or without radiation), DCIS has a local recurrence rate of about 10 percent, half of which are invasive cancers. Mortality rates for DCIS patients with recurrent disease run at approximately one percent. To prevent the 10 percent incidence of local recurrence, and ultimately to prevent the one percent mortality rate, total mastectomy has therefore been recommended by certain practitioners for all DCIS patients. It would be desirable, however, to identify prognostic markers that would indicate which DCIS tumors were likely to recur. A researcher attempting to locate such markers that might be found on the tumor cells themselves could advantageously interact with the systems and methods of the present invention. This example describes how such an encounter could take place.

A researcher may initially wish a panel of DCIS tissue samples to be assembled so that she can screen a number of samples to look for genetic overexpression or underexpression or related differences in mRNA or protein production. Her basic requirement, then, is an assembly of samples of female breast tissue exhibiting DCIS. Recognizing that DCIS is often a condition found in association with invasive breast cancer, she may wish to select only those samples that are “pure” DCIS, without associated invasive disease and without other malignant or pre-malignant conditions such as lobular carcinoma-in-situ. She could enter these requests at a data entry terminal or over the internet, specifying that the tissue array comprise female breast tissue samples whose pathology was only DCIS and not invasive cancer and not lobular carcinoma-in-situ. In Boolean terms, such a request could be phrased as “DCIS andnot (invasive carcinoma or lobular carcinoma-in-situ).” Alternatively, the request could be entered via a dropdown list selection, or via any other data entry means available in the art.

In response to this request, a set of samples within the tissue repository conforming to the request could be selected, according to the systems and methods of the present invention. The system could respond by indicating to the researcher how many samples conforming to her request would be available and the system could further offer her guidance about whether the available number would provide her with statistically significant data. If an inadequate number of samples is available, the researcher could be prompted to broaden her search by eliminating certain limitations or by changing the search terms. If an excessive number of samples is available, the researcher could be prompted to narrow her search by entering additional limitations. Additional limitations could include pathological criteria or clinical specifiers that segregate tissue samples according to criteria selected from the clinical information associated with a particular sample. For example, the researcher could limit her sample selection by specifying that the samples only be derived from premenopausal women, or by requesting that selected samples be derived from women with no history of hormone or birth control pill treatment. Alternatively, to limit the number of samples included in an array, the researcher could direct the system to perform a totally random selection of the identified samples. As another option, the researcher could direct the system to select samples so that they reflect a representative range of certain criteria, for example a wide range of ages, or a wide range of height to weight ratios. The researcher could introduce a variety of other demographic or other clinical limitations, either to restrict the number of samples selected by the system, or to ensure that a certain range of demographic variability exists in the sample pool. Representative variables could include the presence or absence of a family history for breast cancer or other cancers, the age at menarche, the age at menopause, the age at first pregnancy, the history of hormone use including birth control pills, height and weight, exposure to cigarette smoke or radiation, and racial or national origin data.

If the researcher were concerned that atypical ductal hyperplasia samples were erroneously diagnosed as DCIS, she could request that an image of each sample be provided online for her inspection, or she could request that a certain subset of images from samples specifically or randomly selected be provided for her inspection. Using the images, she could use her own judgment to decide whether the diagnosis of DCIS associated in the system with each sample was accurate. Alternatively, referring to the image, she could identify on the image itself a paradigmatic exemplification of DCIS and ask the system to find more samples whose images corresponded to the paradigm she had selected.

Since DCIS specimens in some cases will be derived from total mastectomy specimens, there may be normal breast tissue available from certain of the patients who provided DCIS samples. There may also be normal breast tissue available that was removed from patients with no pathological breast diagnosis, for example patients undergoing breast reduction surgery. The researcher might ask to have a panel assembled that would include, in addition to DCIS tissue, normal breast tissue from DCIS patients (either matched or unmatched to the DCIS samples provided), and normal breast tissue from control patients without pathological breast diagnosis. The availability of unaffected tissue from the DCIS patient could allow the researcher to identify genes that are upregulated or downregulated in breast tissue that is clinically normal but that is derived from a patient who has demonstrated a propensity for developing breast cancer. The availability of normal breast tissue from patients without pathological breast diagnosis may permit controls to be compared to pathological samples so that variations specifically associated with pathology may be more readily isolated.

According to the systems and methods of the present invention clinical data that would include information derived from a patient's medical record may be arranged in a database and related to tissue samples derived from a particular patient. Besides information gathered contemporaneously with the acquisition of the tissue sample, this database of clinical data may contain information that is updated following tissue sample acquisition. Information about the follow-on clinical course in each patient whose tissue sample is being screened could be especially important to a researcher. The researcher might, for example, be interested in assembling a panel where an initial diagnosis of DCIS was locally treated with either wide excision or wide excision plus radiation therapy, and where information is available to indicate whether there has been local recurrence following the initial treatment. In light of the restrictions imposed by anonymization, useful follow-on data may be acquired by the system when a tissue sample is acquired at a second surgical procedure for a DCIS patient, where this second tissue sample would represent local recurrence. At the time the second sample is obtained, associated clinical information would also be acquired that would include the interval between the first sample excision and this second excision. The second tissue sample and the clinical information related to it could be linked within the database to the initial tissue sample and its clinical information, thereby allowing the researcher interested in the first tissue sample to have access to follow-on information about the source patient.

The system, interacting with the researcher, could select samples according to availability of follow-on information. To initiate such a process, for example, the researcher could initially specify that the desired DCIS samples be collected from patients who had not undergone mastectomy (if the DCIS was initially treated by mastectomy, there would be no breast tissue left in which to identify local recurrence). Such a query, in Boolean terms, would add the limitation “andnot mastectomy” to the other Boolean terms used above. If follow-on clinical information about the DCIS patients is available in the database, it could then be referenced by other aspects of the researcher's request. For example, a request term could be entered that allowed the researcher to seek samples only from those patients for whom follow-on data is available The researcher could then limit her requests to that pool of samples, asking within that pool for DCIS tissue from patients who have had subsequent recurrences, who are recurrence-free or both. As a further limitation, the researcher could request DCIS samples from those patients experiencing local recurrence based on whether those local recurrences were invasive or not. Or, as another limitation, she could specify that she was interested in DCIS samples from patients who have follow-on information about local recurrence, and further she could base her selection on whether the patient had undergone wide excision alone or wide excision with radiation. To summarize these options, a researcher employing the systems and methods of the present invention could then order a sample array by specifying her interest in DCIS samples taken from breast conservation patients who have undergone either wide excision or wide excision with radiation, and she could further specify that her selected DCIS samples would/would not be derived from patients with local recurrence, and if DCIS samples from local recurrence patients were included, that the local recurrences would/would not be invasive cancer.

A Boolean representation of part of the query string discussed above could include the following terms: ((DCIS) andnot (“invasive cancer”) andnot (lobular)) and ((“wide excision” or “wide excision with radiation”) andnot (mastectomy)) and (follow-on data available) and (“local recurrence” or “no local recurrence”). A further logical term might be needed: for example, the formulation IF “local recurrence,” THEN (“invasive cancer” or “DCIS”) may indicate to the system that if local recurrence is present, then samples wherein the recurrence comprises either invasive cancer or DCIS would be acceptable. As an alternative to Boolean terms and logic, the researcher could specify the samples she wants by entering into the system information according to certain query categories, such as pathological information, treatment information, clinical information, follow-on information, etc. The researcher could be guided to provide information in these or other categories by prompts or menus provided by the system. For example, the researcher could input initially pathology information, such as tissue type, primary diagnosis, and pathology limitations. In this case, the researcher could respond to the system's queries as follows: 1. source=breast; 2. primary diagnosis=DCIS; 3. limitations=no invasive carcinoma, no lobular carcinoma. These parameters could be input in response to queries or dropdown prompts provided by the system. For example, in response to the specified source “breast,” the system could formulate a dropdown list including the varieties of breast pathology, from which the researcher would select DCIS. Likewise, after DCIS has been selected, a dropdown list could be provided that offers a selection of limitations and that allows the researcher to specify a new limitation not included on the list (e.g., “no Paget's disease”). Following the entry of these data, the researcher could then input parameters based on the type of surgery or other treatment that the sample patient has undergone: 1. surgical procedure=wide excision or biopsy; 2. limitations=no mastectomy samples. The researcher could, in addition, input parameters based on the clinical variables she wishes to use as selectors. For example, to obtain a certain selection of tissue samples, the researcher could input the following clinical and demographic limitations: 1. sex=female; 2. age=20-80; 3. menarche=13+/−3 years; 4. menopause=52+/−5 years; 5. height/weight=random selection; 6. prior hormones=no; 7. family history=no. As shown here, tissue samples would be selected that are derived from a wide age range of patients, with fairly typical data for menarche and menopause, with no prior hormones and no breast cancer family history. This exemplary request profile would also direct the system to select samples from patients so that height and weight distribution is provided for randomly, as shown here, or so that a representative group of small, medium and large sized women are provided. If outcome information is available, the researcher could further select samples from those patients for whom such follow-on information exists. She could then provide limitations to select samples from that pool of patients: 1. follow-on information yes; 2. local recurrence information yes; 3. outcome=local recurrence or no local recurrence; 4. local recurrence outcome=invasive cancer or DCIS. Such a request would select samples from patients having follow-on information where information about local recurrence is recorded, and would gather samples from that pool that have data about local recurrence (whether present or absent) and, if local recurrence is present, that have data about whether the recurrence is invasive cancer or DCIS. Entering criteria pertaining to outcomes would allow samples to be selected on the basis of what happened to the patient after the procedure from which her sample was obtained.

As an alternative approach, a researcher could request of the system samples of invasive breast cancer and specify that these samples be derived from patients who had a previous diagnosis of DCIS without invasive disease in the same breast. If the samples are derived from a mastectomy specimen, she could ask also for matched normal tissue controls from the same patient, perhaps screening out those patients with prior radiation therapy following wide excision'so as to eliminate artifacts related to radiation exposure. The researcher could furthermore ask for a set of control samples of invasive breast cancer specimens derived from patients without history or present diagnosis of DCIS. Using this approach, a researcher could examine the invasive breast cancer specimens in the patients with previous DCIS and determine which genes are upregulated or downregulated, comparing those findings with the control findings. Since the progression of disease from noninvasive intraductal carcinoma to invasive ductal carcinoma may, at a cellular level, involve alteration or transformation of gene expression, examining tissues from invasive local recurrences may not yield enough useful information related to DCIS prognostic markers. However, obtaining an array of invasive recurrence samples using these parameters may allow a researcher who has already carried out studies such as those described above on DCIS samples to crosscheck her findings by examining tissues that have demonstrated their ability to recur as invasive cancer in a treated DCIS patient.

By properly specifying the parameters according to which a tissue array of DCIS specimens would be selected using the systems and methods of the present invention, the researcher may be able to obtain data about genetic or expressive profiles that could have prognostic significance for ductal carcinoma-in-situ. For example, the researcher might be able to identify a marker that indicates low probability of local recurrence, such a marker being found in DCIS samples from “wide excision only” patients without local recurrences. Or, for example, the researcher might be able to identify a marker that indicates particular radiation sensitivity, such a marker being found in DCIS samples from “wide excision only” patients who recurred, and also being found in DCIS samples from “wide excision plus radiation” patients who did not recur. As another example, the researcher might be able to identify a marker that indicates a particularly aggressive type of DCIS, this marker being found in DCIS samples from either “wide excision only” patients or “wide excision plus radiation” patients who recurred locally especially quickly, and/or who developed invasive local recurrences. As is evident to those of ordinary skill in the art, identifying these types of markers could contribute significantly to making therapeutic decisions. A patient, for example, with the aggressive DCIS marker would preferentially be a candidate for total mastectomy, despite her having non-invasive disease initially. On the other hand, a patient with a marker indicating a more indolent type of DCIS would be a good candidate for breast conservation. Identifying a marker that indicated either increased or decreased radiation sensitivity would guide decision making about whether to treat the affected breast with radiation.

The above example is presented for illustrative purposes only. Other variations and utilizations of the systems and methods of the present invention will be apparent to those of ordinary skill in the art and may be carried out using no more than routine experimentation.

What is claimed is:

1. A computer implemented method for providing a biological material or a derivative product thereof to a user, the method comprising:
receiving a query from a user, which identifies at least one desired characteristic of the biological material;
identifying a biological material that has the at least one characteristic;
receiving specification of the format for the biological material; and
providing the at least one biological material or derivative product, in the specified format to the user.

2. The method of claim 1, wherein the characteristic comprises clinical data.

3. The method of claim 2, wherein the clinical data is selected from the group consisting of: familial data, clinical history data, medication data, disease data, treatment history data, demographic data, laboratory report data, pathology report data, histology report data, and outcome report data.

4. The method of claim 1, wherein the characteristic comprises image data.

5. The method of claim 1, wherein the characteristic comprises molecular profile data (RNA expression, protein expression, metabolite levels).

6. The method of claim 1, wherein the format is selected from the group consisting of: fresh, frozen or paraffin embedded.

7. The method of claim 1, wherein the derivative product is selected from the group consisting of: a tissue section, histocore, cell section, isolated cell(s), DNA, RNA or protein.

8. The method of claim 1, wherein the query is received over a network.

9. The method of claim 8, wherein the network comprises the Internet.

10. The method of claim 8, further comprising transmitting a user interface including instructions for receiving user input defining the query.

11. The method of claim 10, wherein the instructions comprise HTML (HyperText Markup Language).

12. The method of claim 1, wherein the biological material is stored in a repository.

13. The method of claim 1, wherein clinical pathological data is provided to the user.

14. A computer implemented method for providing clinical data associated with patient samples, the method comprising:
(a) receiving from a user a query, which identifies at least one desired characteristic of the biological material;
(b) identifying biological material that has the at least one characteristic; and
(c) providing to the user the clinical data associated with the biological material identified in step (b).

15. The method of claim 14, wherein the clinical data is selected from the group consisting of: familial data, clinical history data, medication data, disease data, treatment history data, demographic data, laboratory report data, pathology report data, histology report data, an outcome report data or an image of the biological material.

16. The method of claim 14, wherein the query is received over a network.

17. The method of claim 16, wherein the network comprises the Internet.

18. The method of claim 16, further comprising transmitting a user interface including instructions for receiving user input defining the query.

19. The method of claim 18, wherein the instructions comprise HTML (HyperText Markup Language).
